(19) United States
(12) Patent Application Publication
(10) Pub. No.: US 2015/0112207 A1
(43) Pub. Date: Apr. 23, 2015

(54) PULSE WAVE SENSOR AND BIOLOGICAL INFORMATION MEASURING DEVICE USING THE SAME

(71) Applicant: SEIKO EPSON CORPORATION, Tokyo (JP)

(72) Inventor: Masashi INOUE, Sakata-shi (JP)

(21) Appl. No.: 14/515,750

(22) Filed: Oct. 16, 2014

(30) Foreign Application Priority Data
Oct. 23, 2013 (JP) 2013-219891

(51) Int. Cl. A61B 5/024; A61B 5/00

(52) U.S. Cl. CPC A61B 5/02444; A61B 5/0059; A61B 5/6824
USPC 600/479

(57) ABSTRACT

A pulse wave sensor includes a sensor substrate that has a first main surface and a second main surface that are in a front-back relationship with each other, has formed therein a first opening part and a second opening part that pass through the first and second main surfaces, and includes a non-transparent portion at least between the first opening part and the second opening part, a light emitting element disposed within the first opening part and having a light emitting surface, and a reflected light detection element having a detection surface on which light reflected after being emitted from the light emitting surface of the light emitting element is incident.

### BACKGROUND

1. Technical Field

The present invention relates to a pulse wave sensor that optically measures a pulse wave of a subject, and a biological information measuring device or the like that uses such a pulse wave sensor.

2. Related Art

When the heart contracts and blood is ejected from the left ventricle into the aorta, the pressure in the aorta changes, and this change in pressure is further conveyed to the peripheral arteries. The waveform obtained by capturing the change in the pulsation of blood vessels (pulse) accompanying this ejection of blood from the heart is referred to as a pulse wave. Measuring the change in capacity of the peripheral vascular system rather than the motion of the heart itself is said to indirectly yield information having similar significance to an electrocardiogram.

In pulse wave measurement utilizing photoelectric conversion, a pulse wave sensor having a light emitting element and a light receiving element is attached to the measurement site, being the subject's wrist, finger, earlobe or the like. In this state, light having a wavelength that is readily absorbed by blood is irradiated toward the measurement site from the light emitting element, and light reflected by body tissue or light transmitted through body tissue is incident on the light receiving element after been subject to variation in attenuation caused by blood flowing through the blood vessels. Furthermore, the light receiving element photoelectrically converts the incident light to generate a pulse wave measurement signal, which is an electrical signal.

Here, in the case where the intensity of light irradiated from the light emitting element and incident on the light receiving element without being mediated by body tissue is high, there is a problem in that the signal-to-noise (S/N) ratio decreases and pulse wave measurement cannot be accurately performed. Accordingly, in order to improve the accuracy of pulse wave measurement, light that is incident on the light receiving element directly from the light emitting element needs to be blocked.

As related technology, JP-A-2013-63203 (paras. 0006-0007; FIG. 4) discloses a pulse wave sensor that aims to accurately measure the pulse wave when the subject is active. This pulse wave sensor is provided with an optical sensor part that acquires pulse wave data by detecting the intensity of light that has been irradiated onto a living body from the light emitting part and transmitted through the living body with a light receiving part. The optical sensor part has a box-shaped casing and a light shielding wall that divides the casing into a first area in which the light emitting part is mounted, and a second area in which the light receiving part is mounted.

However, in JP-A-2013-63203, the thickness of the pulse wave sensor cannot be reduced, due to the optical sensor part having the box-shaped casing and the light shielding wail dividing the casing into the first area in which the light emitting part is mounted and the second area in which the light receiving part is mounted.

### SUMMARY

In view of the above, an advantage of some aspects of the invention is to reduce the thickness of a pulse wave sensor. Also, a further advantage of some aspects of the invention is to provide a biological information measuring device or the like that uses such a pulse wave sensor.

A pulse wave sensor according to one aspect of the invention includes a sensor substrate that has a first main surface and a second main surface that are in a front-back relationship with each other, has formed therein a first opening part and a second opening part that pass through the first and second main surfaces, and includes a non-transparent portion at least between the first opening part and the second opening part; a light emitting element disposed within the first opening part and having a light emitting surface; and a reflected light detection element having a detection surface on which light reflected after being emitted from the light emitting surface of the light emitting element is incident.

According to this aspect of the invention, the sensor substrate includes a non-transparent portion at least between the first opening part and the second opening part, thus allowing this non-transparent portion to function as a light shielding wall that shields light between the light emitting element and the reflected light detection element. Accordingly, a special light shielding component does not need to be provided in the sensor substrate in order to shield light between the light emitting element and the reflected light detection element, thus enabling the thickness of the pulse wave sensor to be reduced, and cost cutting to be achieved with respect to the pulse wave sensor.

Here, the sensor substrate desirably has non-transparency of a degree such that the reflected light detection element does not react to light from the light emitting surface of the light emitting element that is incident on the detection surface of the reflected light, detection element via the sensor substrate. In this case, the possibility of the S/N ratio of the pulse wave measurement signal deteriorating due to light that is incident on the detection surface of the reflected light detection element from the light emitting surface of the light emitting element via the sensor substrate is eliminated.

In the above, a configuration may be adopted in which the sensor substrate includes a reflective layer formed on the lateral surface of the first or second opening part. Light that is incident, on the detection surface of the reflected light detection element from the light emitting surface of the light emitting element via the sensor substrate can be reduced, as a result of the reflective layer reflecting light that is emitted from the light emitting element.

Alternatively, a configuration may be adopted in which the sensor substrate includes a plating layer formed on the lateral surface of the first or second opening part. Light that is incident on the detection surface of the reflected light detection element from the light emitting surface of the light emitting element via the sensor substrate can be reduced, as a result of the plating layer reflecting or attenuating light that is emitted from the light emitting element. In this case, the plating layer can be formed on the lateral surface of the first opening part or the second opening part of the sensor substrate, utilising a manufacturing process of a typical double-sided wiring board having through holes.

The light emitting surface of the light emitting element desirably does not protrude out beyond the second main surface of the sensor substrate. In this case, the spreading of light that is emitted from the light emitting surface of the light emitting element is suppressed, thus enabling the amount of light that is incident on the detection surface of the reflected light detection element after being reflected by objects other than body tissue to be reduced.

Also, the light emitting element may further have a lens part disposed on the light emitting surface so as to protrude out beyond the second main surface of the sensor substrate. Light that is emitted from the light emitting surface of the light emitting element is converged as a result of providing the lens part, and the thickness of the sensor substrate does not need to be increased in order to provide the lens part, as a result of configuring the lens part to protrude out beyond the second main surface of the sensor substrate.

A plurality of terminals of the light emitting element and a plurality of terminals of the reflected light detection, element may be respectively connected by solder to a plurality of electrodes formed on the first main surface of the sensor substrate. In this case, general surface mount technology can be used.

Alternatively, the reflected light detection element may be mounted on the first main surface of the sensor substrate, using an ACF (anisotropic conductive film) or an ACP (anisotropic conductive paste). In this case, even if the interval between terminals aligned in the horizontal direction is small, the reflected light detection element can be mounted without the terminals short circuiting.

Also, an ACF (anisotropic conductive film), an ACP (anisotropic conductive paste), an NCF (non-conductive film) or an NCP (non-conductive paste) may be filled between, the reflected light detection element and the first main surface of the sensor substrate, at a periphery of the second opening part. In this case, the gap between the reflected light detection element and the first main surface of the sensor substrate is closed off, thus enabling the S/N ratio of the pulse wave measurement signal to be improved by blocking light leaking to the detection surface of the reflected light detection element from the first main surface side of the sensor substrate.

Furthermore, a biological information measuring device according to one aspect of the present invention includes any of the pulse wave sensors described above; a transparent substrate provided opposing the second main surface of the sensor substrate; a circuit part that measures a pulse interval or a pulse rate, based on a pulse wave measurement signal that is output from the pulse wave sensor; a display part that displays a measurement result of the circuit part; a casing that supports the display part on a first surface and the transparent substrate on a second surface having a front-back relationship with the first surface, and has built therein the pulse wave sensor and the circuit part. According to this aspect of the present invention, the pulse wave sensor built into the casing of the biological information measuring device is thin, thus enabling the thickness of the biological information measuring device to also be reduced.

### BRIEF DESCRIPTION OF THE DRAWINGS

The invention will be described with reference to the accompanying drawings, wherein like numbers reference like elements.

FIG. 1 is a partial cross-sectional view showing a pulse wave sensor and a periphery thereof according to one embodiment of the present invention.

FIG. 2 is an assembly diagram of the pulse wave sensor shown in FIG. 1.

FIG. 3 is a schematic diagram showing an external appearance of a biological information measuring device that uses the pulse wave sensor shown in FIG. 1.

FIG. 4 shows an exemplary configuration of the biological information measuring device that uses the pulse wave sensor shown in FIG. 1.

### DESCRIPTION OF EXEMPLARY EMBODIMENTS

Hereinafter, embodiments of the invention will be described in detail with reference to the drawings. Note that the same reference signs are given to constituent elements that are the same, and redundant description will be omitted.

A pulse wave sensor according to the present invention is, for example, incorporated for use into a wristwatch type biological information measuring device provided with a pulse wave measurement function and a clocking function or a biological information measuring device having a tablet form. Hereinafter, a pulse wave sensor incorporated into a wristwatch type biological information measuring device will be described as an example.

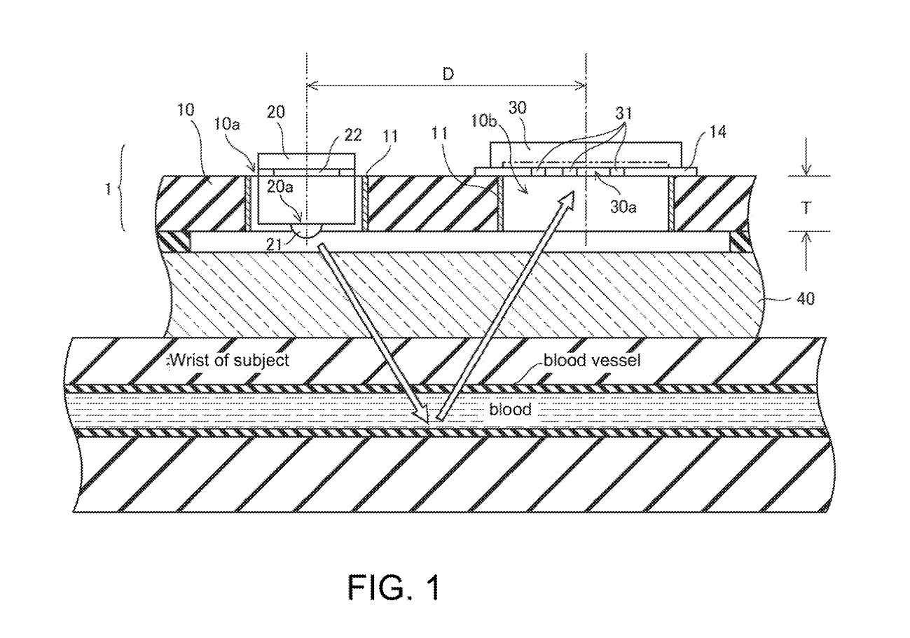

FIG. 1 is a partial cross-sectional view shows a pulse wave sensor and a periphery thereof according to one embodiment of the present invention. As shown in FIG. 1, a pulse wave sensor 1 includes a sensor substrate 10, a light emitting element 20, and a reflected light detection element 30. The sensor substrate 10 has a first main surface (upper surface in the diagram) and a second main surface (lower surface in the diagram) that oppose each other, and a first opening part 10a and a second opening part 10b that pass through, the first and second main, surfaces are formed therein. The first main surface and the second main surface are in a front-back relationship. A glass epoxy substrate, for example, can be used as the sensor substrate 10.

The light emitting element 20 is mounted on the first main surface of the sensor substrate 10, has a light emitting surface 20a inserted within the first opening part 10a, and emits light from the light emitting surface 20a. The light emitting surface 20a is disposed within the first opening part 10a. An LED (light emitting diode) that emits light having an intensity corresponding to the drive current that is supplied, for example, can be used as the light emitting element 20. A color (e.g., blue) having a wavelength that is readily absorbed by blood is suitable as the emission color. The light emitting element 20 may further have a lens part 21 disposed on light emitting surface 20a. Providing the lens part 21 enables light that is emitted from light emitting surface 20a of the light emitting element 20 to be converged.

The reflected light detection element 30 is mounted on the first main surface of the sensor substrate 10, and has a defection surface 30a onto which light emitted from light emitting surface 20a of the light emitting element 20 and reflected on the second main surface side of the sensor substrate 10 is incident after passing through the second opening part 10b. As shown in FIG. 1, the detection surface 30a of the reflected light detection element 30 faces the sensor substrate 10 side (downward in the diagram). The detection, surface 30a can be disposed within the second opening part 10b, on the first main surface, or so as to be separated from the sensor substrate 10. The reflected light detection element 30 generates a pulse wave measurement signal, which is an electrical signal, by photoelectrically converting light that is incident on the detection surface 30a. A PD (photo-diode) that outputs a detection current, corresponding to the intensity of incident light, for example, can be used as the reflected light detection element 30.

A transparent substrate 40, which is part of the biological information measuring device, is provided, opposing the second main surface of the sensor substrate 10. Glass or an acrylic, for example, can be used as the material of the transparent substrate 40. The sensor substrate 10 and the transparent substrate 40 are supported by a casing of the biological information measuring device. When the biological information measuring device is attached to the subject's wrist, the transparent substrate 40 faces the subject's wrist. Accordingly, light emitted from light emitting surface 20a of the light emitting element 20 is incident on the subject's wrist.

The light incident on the subject's wrist passes through the subject's outer skin, and reaches the blood vessels within the inner skin which is below the outer skin. Part of the light that reaches the blood vessels is absorbed by the blood flowing through the blood vessels. On the other hand, part of the light that was not absorbed by blood, out of the light that reached the blood vessels, reaches the detection surface 30a of the reflected light detection element 30 as reflected light after being scattered by body tissue, or the like.

Here, the subject's blood vessels (particularly the arteries) repeatedly expand and contract in synchronous with the cardiac cycle. Accordingly, the attenuation of light by blood flowing through the blood vessels varies temporally at the same cycle as the cycle of expansion and contraction of the blood vessels. In view of this, the subject's pulse wave can be measured by detecting the intensity of light that has passed through the subject's blood vessels. The pulse wave measurement signal that is output from the reflected light detection element 30 will have a component representing the change in capacity of blood vessels at the measurement site.

In the case where the intensity of light irradiated, from the light emitting element 20 and incident on the reflected light detection element 30 without being mediated by body tissue is high, the S/N ratio of the pulse wave measurement signal decreases, and pulse wave measurement cannot be accurately performed, as is evident from the measurement principles of such a pulse wave sensor 1. Accordingly, in order to improve the accuracy of pulse wave measurement, light that is incident on the reflected light detection element 30 directly from the light emitting element 20 needs to be blocked.

Thus, in the present embodiment, the sensor substrate 10 includes a non-transparent portion at least between the first opening part 10a and the second opening part 10b. The non-transparent portion of the sensor substrate 10 thereby functions as a light shielding wall that shields light between the light emitting element 20 and the reflected light detection element 30. Accordingly, a special light shielding component (e.g., the box-shaped, casing and light shielding wall in JP-A-2013-63203) does not need to be provided in the sensor substrate 10 in order to shield light between the light emitting element 20 and the reflected light detection element 30, thus enabling the thickness of the pulse wave sensor 1 to be reduced, and cost cutting to be achieved with respect to the pulse wave sensor.

Here, the sensor substrate 10 desirably has non-transparency of a degree such that the reflected light-detection element 30 does not react to light from the light emitting surface 20a of the light emitting element 20 that is incident on the detection surface 30a of the reflected light detection element 30 via the sensor substrate 10. In this case, the possibility of the S/N ratio of the pulse wave measurement signal deteriorating due to light from the light emitting surface 20a of the light emitting element 20 being incident on the detection surface 30a of the reflected light detection element 30 via the sensor substrate 10 is eliminated.

In order for the sensor substrate 10 to function as a light shielding wall, the sensor substrate 10 itself may have low transmissivity with respect to light that is emitted from the light emitting element 20. Alternatively, as shown in FIG. 1, the sensor substrate 10 may include a reflective layer or plating layer 11 formed on the lateral surface of the first opening part 10a or the second opening part 10b. As a result of the reflective layer reflecting light that is emitted from the light emitting element 20, or the plating layer reflecting or attenuating light that is emitted, from the light emitting element 20, light that is incident on the detection surface 30a of the reflected light detection element 30 from the light emitting surface 20a of the light, emitting element 20 via the sensor substrate 10 can be reduced.

Generally, in the case of a double-sided wiring substrate, a method of forming a metal layer made of copper (Cu) or the like by electroless plating on the wail surface of the through holes is employed. Accordingly, a metal reflective layer or plating layer 11 made of copper (Cu), copper alloy or the like can be formed on the lateral surface of the first opening part 10a or the second opening part 10b of the sensor substrate 10, utilizing a manufacturing process of a typical double-sided wiring substrate having through holes. For example, nickel silver that includes 50% to 70% of copper (Cu), 5% to 30% of nickel (nickel) and 10% to 30% of zinc (Zn) may be used as the material, of the reflective layer or plating layer 11.

In FIG. 1, a distance D between the central axis of the light emitting element 20 and the central axis of the reflected light detection element 30 is 2 mm, for example, and a thickness T of the sensor substrate 10 is 0.65 mm, for example. Here, the light emitting surface 20a of the light emitting element 20 desirably does not protrude out beyond the second main surface of the sensor substrate 10. In this case, the spreading of light that is emitted from the light emitting surface 20a of the light emitting element 20 is suppressed, thus enabling the amount of light that is incident on the detection surface 30a of the reflected light detection element 30 after being reflected by objects (transparent substrate 40, etc.) other than body tissue to be reduced.

In the case where, however, the light emitting element 20 has the lens part 21, the lens part 21 may project out beyond the second main surface of the sensor substrate 10, because light that is emitted from the light emitting surface 20a of the light emitting element 20 is converged by the lens part 21. The thickness of the sensor substrate 10 thereby does not need to be increased in order to provide the lens part 21.

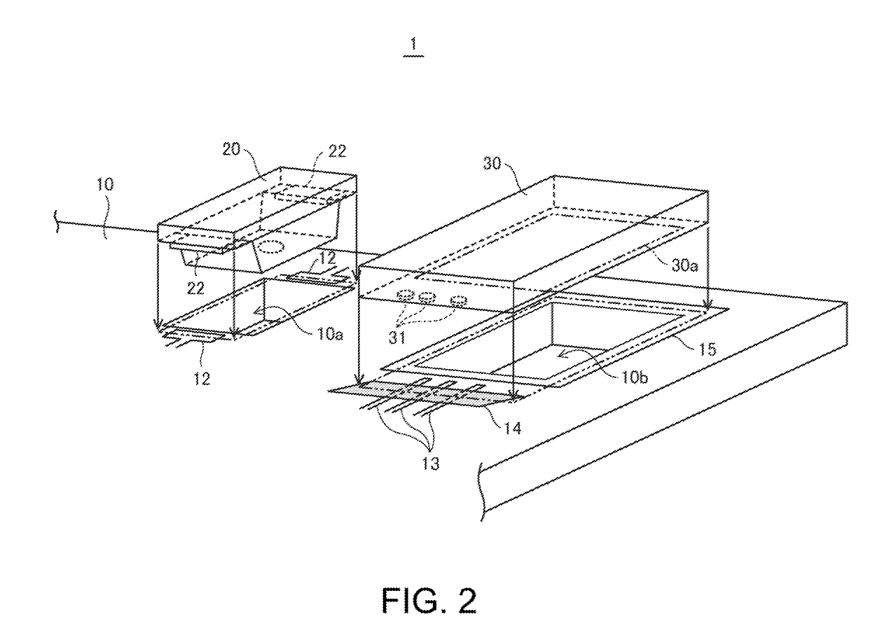

FIG. 2 is an assembly diagram of the pulse wave sensor shown in FIG. 1. As shown in FIG. 2, the light emitting element 20 has a plurality of terminals 22. Also, the reflected light detection element 30 also has a plurality of terminals 31. The plurality of terminals of the light emitting element 20 and the reflected light detection element 30 may be respectively connected by solder to a plurality of electrodes 12 and 13 formed on the first main surface of the sensor substrate.

In this case, general surface mount, technology can be used. That is, the light emitting element 20 and the reflected light detection element 30 are mounted using a chip mounter, after performing solder printing on the sensor substrate 10 using a cream solder printing machine, and the sensor substrate 10 on which the light emitting element 20 and the reflected light detection element 30 are mounted is placed in a reflow furnace and the solder is melted, such that the light emitting element 20 and the reflected light defection element 30 are fixed to the sensor substrate 10 after cooling.

Alternatively, the reflected light detection element 30 may be mounted on the first main surface of the sensor substrate 10 by flip chip bonding, using an ACF (Anisotropic Conductive Film) or ACP (Anisotropic Conductive Paste) 14 shown in FIG. 2.

An ACF is a film produced by forming a substance obtained by mixing fine conductive particles with a thermosetting resin into a film. Here, the conductive particles are spheres of about 3 μm to 5 μm in diameter in which a nickel layer as the innermost layer, a metal plated layer, and an insulating layer as the outermost layer are stacked, for example. Also, an ACP is a paste produced by diffusing conductive particles throughout a resin, and differs from the ACF in not being formed as a film.

In the case of using an ACF for mounting the reflected light detection element 30, the ACF is inserted between the terminal 31 of the reflected light detection element 30 and the electrode 13 of the sensor substrate 10, and when pressure is applied to the reflected light detection element 30 with a pad made of rubber or the like having elasticity while heat is applied with a heater or the like, pressure is exerted on only the portion of the film that is in contact with the raised part of the terminals 31.

As a result, the conductive particles dispersed throughout the film overlap while contacting each other and are eventually pressed together, and a conductive path is formed in the vertical direction (direction approximately orthogonal with the first main surface of the sensor substrate 10) as a result of the plating layers within the conductive particles bonding together. The terminals 31 of the reflected light detection element 30 are thereby electrically connected to the electrodes 13 of the sensor substrate 10.

On the other hand, because the insulating layers of the conductive particles in the portion of the film on which pressure was not exerted are maintained, the terminals aligned in the horizontal direction (direction approximately parallel to the first main surface of the sensor substrate 10) remain insulated from, each other. That is, anisotropy having conductivity in the vertical direction and insulation properties in the horizontal direction is realised. Even if the interval between the terminals that are aligned in the horizontal direction is small, the reflected light detection element 30 can thereby be mounted without the terminals short circuiting.

Furthermore, the ACF or ACP 14 may be filled between the reflected light defection element 30 and the first main surface of the sensor substrate 10 so as to extend to the periphery of the second opening part 10b. Alternatively, as shown in FIG. 2, an NCF (Non-Conductive Film) or NCP (Non-Conductive Paste) 15 may be filled between the reflected light detection element 30 and the first main surface of the sensor substrate 10 so as to extend to the periphery of the second opening part 10b. An NCF or NCP is a film or paste for adhering electronic components to a substrate and consists primarily of resin.

The gap between the reflected light detection element 30 and the first main surface of the sensor substrate 10 is thereby closed off, thus enabling the S/N ratio of the pulse wave measurement signal to be improved by blocking light leaking to the detection surface 30a of the reflected light detection element 30 from the first main surface side of the sensor substrate 10. Also, because of their low fluidity, there is no possibility that these films or pastes will flow as far as the detection surface 30a of the reflected light detection element 30.

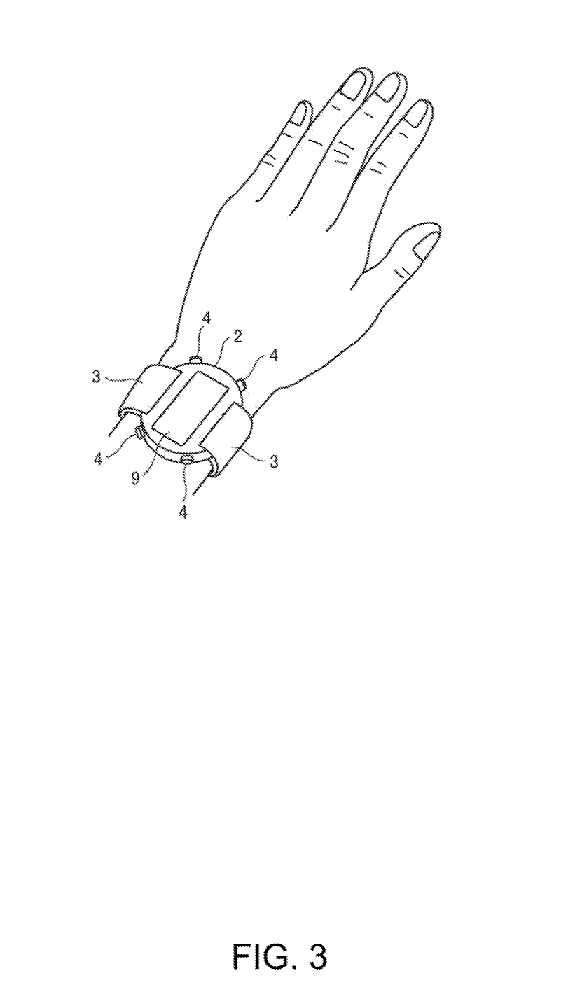

FIG. 3 is a schematic diagram showing an example of the external appearance of a biological information measuring device that uses the pulse wave sensor shown in FIG. 1. This biological information measuring device has a shape modeled on a wristwatch, and has the clocking function of a normal wristwatch in addition to a pulse wave measurement function. A band 3 is attached to a casing 2 of the biological information measuring device, and the biological information measuring device is attached to the subject's body by wrapping the band 3 around the subject's wrist and fastening the band 3.

A plurality of button switches 4 are provided on a peripheral part of the casing 2. Those button switches 4 are used in order to input various commands for starting and stopping pulse wave measurement, resetting the measurement result, setting the time, and the like. The casing 2 supports a display part 9 having a rectangular display surface on a first surface (front surface). The pulse interval or pulse rate is displayed on the display part 9 as a measurement result, or time is displayed similarly to a normal wristwatch. On the other hand, the casing 2 supports the transparent substrate 40 shown in FIG. 1 on a second surface (back surface) that opposes the first surface. The biological information measuring device shown in FIG. 3 is, however, merely one example of a biological information measuring device according to the present embodiment, and a biological information measuring device according to the present embodiment may be attached to sites other than the subject's wrist.

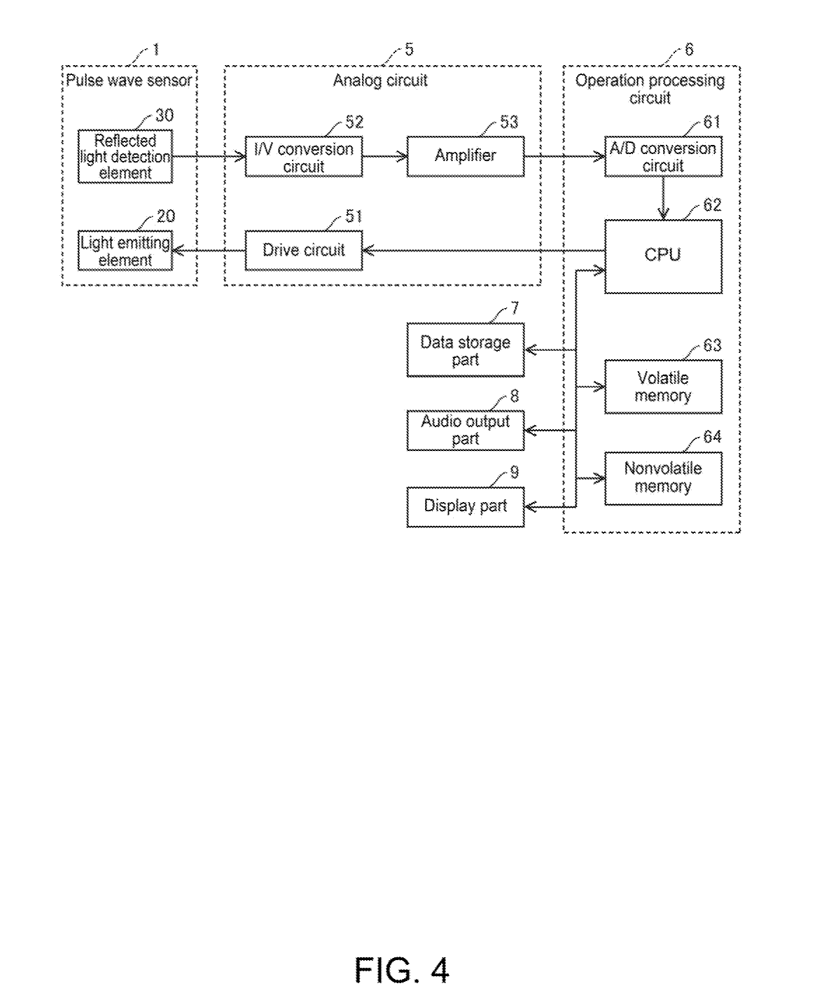

FIG. 4 is a block, diagram showing an exemplary configuration of a biological information measuring device that uses the pulse wave sensor shown in FIG. 1. Note that, in FIG. 4, the configuration relating to the clocking function is omitted. As shown in FIG. 4, the biological information measuring device includes the pulse wave sensor 1, an analog circuit 5, an operation processing circuit 6, a data storage part 7, an audio output part 8, and the display part 9. Here, the constituent elements from the pulse wave sensor 1 to the audio output part 8 are built into the casing 2 shown, in FIG. 3. Also, the analog circuit 5 and the operation processing circuit 6 constitute a circuit part that measures the pulse interval or pulse rate based on the pulse wave measurement signal that is output from the pulse wave sensor 1.

The analog circuit 5 includes a drive circuit 51, an I/V conversion circuit 52, and an amplifier 53. The drive circuit 51 causes the light emitting element 20 of the pulse wave sensor 1 to emit light by supplying a drive current to the light emitting element 20. The I/V conversion circuit 52 converts the detection current (pulse wave measurement signal) that is output from the reflected light detection element 30 of the pulse wave sensor 1 into a voltage, and outputs the voltage to the amplifier 53. The amplifier 53 amplifies the voltage that is output from the I/V conversion circuit 52, and outputs the amplified voltage to the operation processing circuit 6.

The operation processing circuit 6 includes an A/D conversion circuit 61, a CPU (central processing unit) 62, a volatile memory 63 such as a RAM (random access memory), and a nonvolatile memory 64 such as a ROM (read-only memory). The A/D conversion circuit 61 generates pulse wave measurement data by performing analog-to-digital conversion on the voltage that is output from the amplifier 53, and outputs the pulse wave measurement data to the CPU 62.

The CPU 62 operates in accordance with a pulse wave analysis program stored in the nonvolatile memory 64, while utilising the volatile memory 63 as a work area. The pulse wave analysis program causes the CPU 62 to execute poise wave analysis processing. In the pulse wave analysis processing, the CPU 62 measures the pulse interval or pulse rate based on the pulse wave measurement data that is output from the A/D conversion circuit 61, and stores the measurement result in the data storage part 7. Furthermore, the CPU 62 causes the audio output part 8 to output audio notifying that measurement has ended, and causes the display part 9 to display the measured pulse interval or pulse rate.

Next, the operations of the biological information measuring device shown in FIGS. 3 and 4 will be described. When the subject inputs a command to start pulse wave measurement using the button switches 4, the CPU 62 of the operation processing circuit 6 causes the drive circuit 51 to start light emission of the light emitting element 20. Light emitted from the light emitting element 20 is thereby incident on the subject's wrist.

Part of the light that is incident on the subject's wrist is incident on the reflected light detection element 30 after passing through blood vessels and being reflected. The reflected light detection element 30 thereby outputs a pulse wave measurement signal having a component that represents the change in capacity of blood vessels at the measurement site. The pulse wave measurement signal that is output from the reflected light detection element 30 is processed in the analog circuit 5, and converted to pulse wave measurement data by the A/D conversion circuit 61 of the operation processing circuit 6, and the pulse wave measurement data is supplied to the CPU 62.

The CPU 62 measures the pulse interval or pulse rate based on the pulse wave measurement data, and stores the measurement result in the data, storage part 7. For example, the CPU 62 derives the time difference between the latest peak and the previous peak whenever a peak appears in the values of the pulse wave measurement data, and stores this time difference in the data storage part 7 as the pulse interval. Also, the CPU 62 counts, every predetermined time period (e.g., every 1 min.), the number of peaks appearing in the values of the pulse wave measurement data, and stores the counted number of peaks in the data storage part 7 as the pulse rate. Furthermore, the CPU 62 causes the pulse interval or pulse rate stored in the data storage part 7 to be displayed on the display part 9 as the measurement result.

Although a pulse wave sensor incorporated into a wristwatch type biological information measuring device was described in the above embodiments, the present invention is not limited to the embodiments described above. For example, the present invention can also be utilized in a biological information measuring device that takes the subject's finger, earlobe or the like as the measurement site, and a person skilled, in the art will appreciate that numerous modifications can be made within, the technical concept of the invention.

The entire disclosure of Japanese Patent Application No. 2013-219891, filed Oct. 23, 2013 is expressly incorporated by reference herein.

What is claimed is:

1. A pulse wave sensor comprising:
a sensor substrate that has a first main surface and a second main surface that are in a front-back relationship with each other, has formed therein a first opening part and a second opening part that pass through the first and second main surfaces, and includes a non-transparent portion at least between the first opening part and the second opening part;
a light emitting element disposed within the first opening part and having a light emitting surface; and
a reflected light detection element having a detection surface on which light reflected after being emitted from the light emitting surface of the light emitting element is incident.

2. The pulse wave sensor according to claim 1,
wherein the sensor substrate has non-transparency of a degree such that the reflected light detection element does not react to light from the light emitting surface of the light emitting element that is incident on the detection surface of the reflected light detection element via the sensor substrate.

3. The pulse wave sensor according to claim 1,
wherein the sensor substrate includes a reflective layer formed on a lateral surface of the first or second opening part.

4. The pulse wave sensor according to claim 1,
wherein the sensor substrate includes a plating layer formed on a lateral surface of the first or second opening part.

5. The pulse wave sensor according to claim 1,
wherein the light emitting surface of the light emitting element does not protrude out beyond the second main surface of the sensor substrate.

6. The pulse wave sensor according to claim 5,
wherein the light emitting element further includes a lens part disposed on the light emitting surface so as to protrude out beyond the second main surface of the sensor substrate.

7. The pulse wave sensor according to claim 1,
wherein a plurality of terminals of the light emitting element and a plurality of terminals of the reflected light detection element are respectively connected by solder to a plurality of electrodes formed on the first main surface of the sensor substrate.

8. The pulse wave sensor according to claim 1,
wherein the reflected light detection element is mounted on the first main surface of the sensor substrate, using an anisotropic conductive film or an anisotropic conductive paste.

9. The pulse wave sensor according to claim 1,
wherein an anisotropic conductive film, an anisotropic conductive paste, a non-conductive film or a non-conductive paste is filled between the reflected light detection element and the first main surface of the sensor substrate, at a periphery of the second opening part.

10. A biological information measuring device comprising:
the pulse wave sensor according to claim 1;
a transparent substrate provided opposing the second main surface of the sensor substrate;
a circuit part that measures a pulse interval or a pulse rate, based on a pulse wave measurement signal that is output from the pulse wave sensor;
a display part that displays a measurement result of the circuit part; and
a casing that supports the display part on a first surface and the transparent substrate on a second surface having a front-back relationship with the first surface, and has built therein the pulse wave sensor and the circuit part.

11. A biological information measuring device comprising:
the pulse wave sensor according to claim 2;
a transparent substrate provided opposing the second main surface of the sensor substrate;
a circuit part that measures a pulse interval or a pulse rate, based on a pulse wave measurement signal that is output from the pulse wave sensor;
a display part that displays a measurement result of the circuit part; and
a casing that supports the display part on a first surface and the transparent substrate on a second surface having a front-back, relationship with the first surface, and has built therein the pulse wave sensor and the circuit part.

12. A biological information measuring device comprising:
the pulse wave sensor according to claim 3;
a transparent substrate provided opposing the second main surface of the sensor substrate;
a circuit part that measures a pulse interval or a pulse rate, based on a pulse wave measurement signal that is output from the pulse wave sensor;
a display part that displays a measurement result of the circuit part; and
a casing that supports the display part on a first surface and the transparent substrate on a second surface having a front-back relationship with the first surface, and has built therein the pulse wave sensor and the circuit part.

13. A biological information measuring device comprising:
the pulse wave sensor according to claim 4;
a transparent substrate provided opposing the second main surface of the sensor substrate;
a circuit part that measures a pulse interval or a pulse rate, based on a pulse wave measurement signal that is output from the pulse wave sensor;
a display part that displays a measurement result of the circuit part; and
a casing that supports the display part on a first surface and the transparent substrate on a second surface having a front-back relationship with the first surface, and has built therein the pulse wave sensor and the circuit part.

14. A biological information measuring device comprising:
the pulse wave sensor according to claim 5;
a transparent substrate provided opposing the second main surface of the sensor substrate;
a circuit part that measures a pulse interval or a pulse rate, based on a pulse wave measurement signal that is output from the pulse wave sensor;
a display part that displays a measurement result of the circuit part; and
a casing that supports the display part on a first surface and the transparent substrate on a second surface having a front-back relationship with the first surface, and has built therein the pulse wave sensor and the circuit part.

15. A biological information measuring device comprising:
the pulse wave sensor according to claim 6;
a transparent substrate provided opposing the second main surface of the sensor substrate;
a circuit part that measures a pulse interval or a pulse rate, based on a pulse wave measurement signal that is output from the pulse wave sensor;
a display part that displays a measurement result of the circuit part; and
a casing that supports the display part on a first surface and the transparent substrate on a second surface having a front-back relationship with the first surface, and has built therein the pulse wave sensor and the circuit part.

16. A biological information measuring device comprising:
the pulse wave sensor according to claim 7;
a transparent substrate provided opposing the second main surface of the sensor substrate;
a circuit part that measures a pulse interval or a pulse rate, based on a pulse wave measurement signal that is output from the pulse wave sensor;
a display part that displays a measurement result of the circuit part; and
a casing that supports the display part on a first surface and the transparent substrate on a second surface having a front-back relationship with the first surface, and has built therein the pulse wave sensor and the circuit part.

17. A biological information measuring device comprising:
the pulse wave sensor according to claim 8;
a transparent substrate provided opposing the second main surface of the sensor substrate;
a circuit part that measures a pulse interval or a pulse rate, based on a pulse wave measurement signal that is output from the pulse wave sensor;
a display part that displays a measurement result of the circuit part; and
a casing that supports the display part on a first surface and the transparent substrate on a second surface having a front-back relationship with the first surface, and has built therein the pulse wave sensor and the circuit part.

18. A biological information measuring device comprising:
the pulse wave sensor according to claim 9;
a transparent substrate provided opposing the second main surface of the sensor substrate;
a circuit part that measures a pulse interval or a pulse rate, based on a pulse wave measurement signal that is output from the pulse wave sensor;
a display part that displays a measurement result of the circuit part; and
a casing that supports the display part on a first surface and the transparent substrate on a second surface having a front-back relationship with the first surface, and has built therein the pulse wave sensor and the circuit part.
